(19) United States
(12) Patent Application Publication
(10) Pub. No.: US 2022/0042085 A1
(43) Pub. Date: Feb. 10, 2022

(54) System and method of nucleic acid amplification for point of collection

(71) Applicant: Genalyze LLC, Middletown, DE (US)

(72) Inventors: Chen-Yu Liu, Middletown, DE (US); Hui-Ju Tsai, Middletown, DE (US)

(73) Assignee: Genalyze LLC, Middletown, DE (US)

(21) Appl. No.: 17/395,604

(22) Filed: Aug. 6, 2021

Related U.S. Application Data
(60) Provisional application No. 63/063,220, filed on Aug. 7, 2020.
(60) Provisional application No. 63/223,972, filed on Jul. 21, 2021.

(51) Int. Cl. C12Q 1/6844; B01L 3/00; C12Q 1/6876; C12Q 1/6806; G06K 9/00

(52) U.S. Cl. CPC C12Q 1/6844; B01L 3/50; B01L 2300/18; C12Q 1/6806; G06K 9/00134; C12Q 1/6876

(57) ABSTRACT

A system for nucleic acid amplification is to synthesize amplified target nucleic acids or determine the presence of target nucleic acid. The mobile device of the system may be implemented with software for analyzing the reaction or optionally delivering the information of a sample to a cloud. Therefore, the system can provide corresponding genetic information of organism, cancer cells or viruses of interest. The information may include gene expression levels of interest, DNA identity of samples as well as treatment suggestion and professional lists for consulting. The system could also optionally be used with a mobile device to amplify the target nucleic acid for the downstream sequencing or measurement.

### CROSS-REFERENCE TO RELATED APPLICATIONS

This application claims priority from U.S. Provisional Application 63/063,220 entitled “System and method of polymerase chain reaction for point of collection”, filed Aug. 7, 2020, U.S., U.S. Provisional Application 63/223,972 entitled “System and method of nucleic acid amplification for point of collection”, filed Jul. 21, 2020, U.S., and herein incorporated by reference in its entirety.

### TECHNICAL FIELD OF THE INVENTION

The present invention relates generally to system and method of nucleic acid amplification reaction for point-of-collection

### BACKGROUND OF THE INVENTION

There has been a growing interest in the point-of-collection for nucleic acid amplification test for plant pathogen identification, environmental monitoring, sources of foods.

Since all organisms have genetic material such as nucleic acid as well as viruses, the nucleic acids with certain specific sequences could be a signature/genetic feature of a virus or an organism.

Many chemical and biological reactions of interest (e.g., enzymatic amplification reactions) require elevated temperature, so does nucleic acid amplification. There is a need in the art for portable devices having the capability of supplying thermal energy to reactants and allows reactions conducted at different temperatures environments for various reaction stages. Such as polymerase chain reaction (PCR), it needs at least two temperatures for three different stages of polymerase chain reaction-primers and primer annealing, primer extension and denaturation of product DNA from target DNA. Furthermore, when performing the sample preparation or reverse transcription reaction for DNA conversion from RNA, it usually requires different temperatures for samples other than the nucleic acid amplification temperature.

However, the most existing solutions require a thermal cycler. A challenge for processing for a relatively larger amount of samples using a thermal cycler is requirement of a bulky heat exchange device. In term of accessibility, such a bulky device limits its application in a point-of-collection manner.

Because with most of current solutions, only a relatively small number of nucleic acid amplification tests can be carried out in a point-of-collection manner. The precision and accuracy of these tests are relatively low compared to a larger number of samples processed in a regular lab partially due to lacking statistics power.

In addition, it is also difficult for the most current point-of-collection solutions to determine multiple organisms or viruses in a sample without using multiplex PCR. Usage of multiplex PCR may also have certain limitation the number of primers used in one reaction. Because multiplex PCR needs to harmonize reaction conditions and separate results into different optical channels. In addition, the competition between the many primers is a problem. Furthermore, in certain situation, a large number of nucleic acid amplification tests are required to perform at once in a point-of-collection manner such as performing a test for all livestocks in a farm at once. There are a very few solutions being able to handle this type of needs.

In addition, in certain situations, it is desired that nucleic acid amplification is for DNA synthesis and further sequencing in a manner of point-of-collection. One of advantage of using nucleic acid amplification prior to sequencing is to reduce the nucleic acids from background and enrich target nucleic acid sequences.

Furthermore, a DNA synthesis with targeting multiple genome locations may require a relatively larger number of nucleic acid amplification reactions at the same time. To overcome this problem, prior to sequencing, it would be desired if the nucleic acid amplification could be performed in a point-of-collection manner.

Mobile devices with combining nucleic acid amplification technique have the potential of eliminating the need for complicated devices or sensors, which is particular suitable for point-of-collection. The results from nucleic acid amplification could be imaged directly on a mobile device, and the processed data can be stored for tracking or upload to cloud and analyzed directly by an expert in the field such as a physician. Usage of mobile devices with nucleic acid amplification has been the subject of extensive investigation because they have the potential of greatly decreasing the cost and increasing the availability of agriculture pathogen control, environment monitoring and heath care in the world.

Given mobile devices now deeply involve with many people's daily life in US. And most of mobile devices can handle many complex activities such as determining and analyzing the results from nucleic acid amplifications. A user can just purchase a proper nucleic acid amplification kit to amplify their target nucleic acid and use for various purposes. Performing colorimetric-based measurements from a mobile device is one of the most straight forward ways to determine the presence of target nucleic acid. However, it is challenging to use the image directly taking from a mobile device. For instance, one of reasons is different cameras usually generate different RGBA values from an image for the same object.

Many solutions used mobile devices with a thermal cycler and/or microfluidic devices as reaction vessel or have a higher cost.

It is also desirable to have a heat source which is easy to carry and/or scale up for suitably keeping reaction within specific temperature ranges and allowing the quantification of nucleic amplification products or sequencing other than thermal cyclers. It would also be desirable if a mobile device can monitor the temperature of nucleic acid amplification reaction, or determine the nucleic acid synthesis amount without adding extra temperature sensors or controller.

It would be desirable to uses the camera of a mobile device as a colorimeter to determine the presence of target nucleic acid in samples via nucleic acid amplification, and the results obtained from different cameras will have a lower dependency on devices or environments.

It would be desirable to uses a mobile device as a sequencing data processing unit to determine the presence of target nucleic acid in samples via nucleic acid amplification, and sequencing the synthesized DNA with a nanopore sequencer (Genopo: a nanopore sequencing analysis toolkit for portable Android devices, Communication biology, 3, 538 (2020), Genome Med. 7: 99 (2015)).

It would be desirable if nucleic acid of samples can be prepared and amplified in a manner of both higher through-put and point-of-collection. It would be desirable if there is a systematic way to make the nucleic acid tests more accurate and precise.

There is an urgent need for a large number of nucleic acid amplification tests can be perform at once in a resource limited area.

### SUMMARY OF THE INVENTION

The present invention advantageously fills the aforementioned deficiencies by providing system and method of nucleic acid amplification on point-of-collection, which provides a convenience and lower cost solution.

The embodied system may further be characterized by the following illustrative, exemplary, non-limited aspects, features, or steps:

Temperature is an essential factor in many biochemical reactions. For a nucleic acid amplification reaction, it usually requires at least two different temperatures. In addition, sometime, prior to nucleic acid amplification, at a sample preparation stage, a different temperature may be required as well.

The embodied system uses a mean to translocate reaction chambers over different positions on the system, for controlling the temperature of reaction inside the reaction chambers, or for further measurement of nucleic acid amplification result such as taking an image or for sequencing.

In the embodiment, at least one of reaction chambers accommodates a nucleic acid amplification reaction. The reaction chambers shuttle between at least two positions during the biochemical reaction process or product measurement stage.

The two positions may correspond to at least two different heat sources. Thereby, the reaction chambers may have thermal communication with a particular heat sources with a particular temperature and switch to another temperature when have thermal communication with another heat source with another temperature.

Or the reaction chambers may translocate between at least one heat source and at least one position for further measurement of amplified nucleic acid via a detection module. One of non-limited examples of such detection module is a camera on a mobile device, and the measurement is taking at least one image of nucleic acid amplification reaction via the mobile device without using a microfluidic device for nucleic acid amplification.

In one embodiment, the reaction chambers may translocate between two different positions of a system.

In one embodiment, the system has at least one heat source, and a reaction chamber is translocated between the heat source and a position suitable for detection of amplified nucleic acid or a position suitable for collection of amplified nucleic acid.

In one embodiment, the mean drives the translocation of the reaction chambers is a combination of one or more gears, springs or belts.

In one embodiment, the time interval for translocation of reaction chambers is controlled by at least one escapement.

In one embodiment, the translocation mean is merely powered by mechanic force.

In another embodiment, the detection unit is a portable sequencer that links to a mobile device and perform sequencing, in a point-of-collection manner without using a microfluidic devices for the nucleic acid amplification.

In one embodiment, the reaction chamber could be any receptacle with a surface to contact a heat source, and holding reagents and samples, and optionally transparent. The wall of reaction chamber may comprise glass or plastic or metal, which is heat resistant in a temperature range of suitable for biochemical reaction. And the wall of a reaction chamber is eligible to have heat communication with a heat source. Therefore, the temperature within a reaction chamber may be controlled by contacting a heat source with or without a thermal conductive medium. Each reaction chamber has at least one opening to receive reagents and samples while also separating the reagents and samples from the heat source.

A reaction chamber made of a thin layer of heat conductive material may be a suitable choice such as a glass capillary, plastic or metal foil receptacles. In one embodiment, the thickness of the wall of a reaction chamber is at least 0.013 mm.

In one embodiment, the reaction chamber is a 0.2 ml or 0.5 ml PCR tube.

In one embodiment, at least one side of wall for a reaction chamber is transparent. Thereby, the image of the reaction in a chamber may be taken.

In one embodiment, a chamber may have an opening and may be sealed with a liquid with a low evaporating rate or solid lid during reaction. The sealing can be any mean to prevent evaporation of liquid out of a chamber. In one embodiment, the liquid with a low evaporating rate at the temperatures suitable for nucleic acid amplification may be wax, oil, mineral oil, a mineral oil, a silicon oil, or a perfluorinated hydrocarbon. In one embodiment, the opening of a reaction chamber is sealed with a membrane or film.

A test platform comprises of a reaction chamber to hold the nucleic acid amplification reaction, and optionally includes color calibration and/or temperature labels.

In one embodiment, a test platform comprises a cartridge and reaction chambers.

In one embodiment, a colorimetric method may be used to identify any color change due to DNA synthesis or amplification.

In one embodiment, wherein the colorimetric reactive test platform is sensitive to the amount of amplified nucleic acid.

In one embodiment, a color calibration region and temperature label is adjacent to a reaction chamber in a test platform in order to having both images of a reaction chamber and color calibration or temperature label at the same time.

In one embodiment, the color calibration is a reaction chamber containing a predesigned amount of indica or a color label with a predesigned color coordinate.

In one embodiment, wherein the indicia of a sample is proton concentration or metal ion concentration or nucleic acid concentration;

In one embodiment, wherein the modular, colorimetric test platform is a disposable test kit;

In one embodiment, wherein a nanopore sequencer is a detection module, and used for sequencing the amplified nucleic acid in a point-of-collection manner (MARPLE, a point-of-care, strain-level disease diagnostics and surveillance tool for complex fungal pathogens, BMC Biology volume 17, Article number: 65 (2019),).

In one embodiment, a plurality of reaction chambers are hold by one or more receptacles, and the receptacles are driven by one or more motors via one or more of combination of arms, linkages, belts or similar facilities. Thereby, the translocation of reaction chambers via movement of receptacles allows reaction chambers to contact with different heat sources. The heat sources may have different temperatures. Or the translocation disposes one or more reaction chambers to a suitable position for taking an image or for collection of products.

In one embodiment, a plurality of chambers may be situated on a receptacle for reagents and samples, and comprises of a thin glass or plastic or anything suitable for heat communication with a heat source.

In one embodiment, the reaction chamber may have a flat surface at bottom and may connect to a conveyor. The conveyor may shuttle the reaction chamber horizontally over top surface of heat sources. The heat source may also have a flat surface suitable to contact with a reaction chamber. The non-limited examples of a heat source may be a rubber silicon heat pad or polyimide heater with a large surface or a large heat template. Each of heat sources may have different temperatures and remain at a constant temperature during movement of conveyor or contacting with a reaction chamber. The reaction chamber may quickly reach to a specific temperature when contacting with a heat source with the specific temperature. Once the conveyor transfer the reaction chamber over another heat source with another specific temperature, the reaction chamber may also quickly change its temperature accordingly. Thereby, the reaction chamber's temperature may be controlled by driving the conveyor which transfers the reaction chamber over different heat sources, and the biochemical reaction in the reaction chamber may be controlled by driving the conveyor over different heat sources as well. The conveyor may deliver the reaction chamber over a location suitable for taking an image or nucleic acid amplification product collection.

In one embodiment, the reaction chambers are receptacles for samples and reagents. The receptacles have ridged walls/surfaces and at least one surface is transparent to facilitate imaging taking.

In one embodiment, the conveyor may be driven by at least one actuator or motor.

In one embodiment, a receptacle comprises a plurality of reaction chambers situated at a carrier. The carrier comprises at least one motor and a plurality of wheels.

In one embodiment, the receptacle has a flat surface for contacting with at least a heat source having a flat surface. The distance and interval of movement of the carrier is controlled and programmed by an integrated circuit broad. The translocation of receptacle over different heat sources may control the temperature of biochemical reaction inside reaction chambers and the interval of motion may regulate the duration of reactions. Furthermore, the translocation may dispose the receptacle to a proper position for imaging taking or liquid dispensing.

In one embodiment, the bottom and/or side surface of reaction chambers are transparent and allow light to pass through. Thereby, the image sensor or camera may determine the color change inside of reaction chambers.

In one embodiment, a reaction chamber is a glass capillary. The capillary is hold by a receptacle connected to an arm and driven by at least one motor. And the capillary may have fluid communication with reagents and samples in a reservoir. The capillary may draw samples or reagents via capillary action, or connect to a pump or a rubber pipette bulb for liquid dispensing. The capillary may be further sealed with clay, sealant or anything preventing leaking when contacting with heat sources. Or the capillary may be used as a mean to transfer a sample or reagents to a proper receptacle for further reaction steps or/and detection.

In one embodiment, the sealant used in sealing the capillary can be a photopolymer.

In one embodiment, the sealant is a dental composite resin.

In one embodiment, the system comprises a kit for amplification of target nucleic acid sequences, a heat source, a mean for translocation of a reaction chamber relatively to a heat source, a mobile device, and said software installed on said mobile device. The heat source comprises a heating element or heating material and/or a thermal conductive media.

In one of embodiments, said portable, modular, point-of-collection, colorimetric-based system, comprises a mobile device, accessories; wherein said accessories include but not limited to at least one heat source, a mean for translocation relatively to the heat source for a reaction chamber or sample preparation chamber, a nucleic acid amplification kit, a nucleic acid extraction kit/device.

In one embodiment, capillaries are used for samples or reagent dispensing or transferring.

In one embodiment, wherein one optional modular, colorimetric test platform includes a heat source, and an optional light diffuser and/or an optional light-diffusing pathway so as to ensure a uniform and repeatable illumination of at least a desired region of the modular, colorimetric test platform, wherein the light source is one of an internal mobile device flash source, an external LED source or an ambient light source, and optional optical filters and/or lens.

In one embodiment, a mobile device accessory for use in a mobile device—based point-of-collection, colorimetric-based, quantitative measuring system further includes a light source. A light diffuser disposed intermediate the light source and a resident mobile device camera in the mobile device to which the mobile device accessory is linked, in a manner to provide diffuse illumination of a colorimetric test platform when the colorimetric test platform is disposed over the light source.

In one embodiment, the utility of this integrated, test platform is demonstrated by amplifying DNA and/or visually detecting the amplification products. The test platform is particularly suitable for use in the field, in resource-limited regions of the world (where funds and trained personnel are in short supply), in remote areas, and at home. Other heat sources, such as battery-powered sources, solar-powered sources, electric grid power heat sources and other heat sources that derive heat from exothermic reactions are all suitable.

In one embodiment, the nanopore sequencing may be used to identify any target DNA due to DNA synthesis or amplification.

In one embodiment, a sample is collected and loaded into a reaction chamber in the mobile part of a test platform. The mobile part of the test platform is driven by a motor and translocates to a predesigned position. Thereby the reaction chamber on the test platform may contact with a heat source and maintains at a constant temperature or within a narrow range of temperature for a period of time. The temperature or a range of temperature and the time duration is suitable for carrying out a biochemical reaction or a stage of a biochemical reaction. Once the stage of a biochemical reaction is complete, the motor may drive the mobile part of test platform to contact with another heat source for another temperature. In one embodiment, the motor drives the mobile part of test platform to a predesigned position for taking an image or collecting reaction product. In one embodiment, the image is taken by a mobile device and processed locally to determine presence of target nucleic acid, or upload to a cloud for further analysis. In one embodiment, the reaction product is further used for nucleic acid amplification or for sequencing amplified nucleic acid.

In one embodiment, one or more samples are collected through capillaries

In one embodiment, the biochemical reactions include but not limited: cell lysis reaction, DNA denaturation, DNA synthesis, DNA annealing, reverse transcription.

In one embodiment, the system determines the presence of a target nucleic acid sequences in a sample via nucleic acid reaction products with specific primers. In one embodiment, a colorimetric method is used, an image taken by an image sensor for detection of color change in a reaction.

In one embodiment, the system determines the sequence of amplified nucleic acid through a nanopore sequencer.

As known in art, the image associated with the color changes are due to the amplification of target nucleic acid. Usually, the color change of nucleic acid reaction is caused by dye chelating with amplified DNA, fluorescence quenched or activated when target DNA extends in reaction, a pH indicator associated with increasing proton concentration from the reaction as well as some metal ion indicators because of change of free magnesium ion concentration from the reaction.

In one embodiment, wherein the color calibration region maintains a constant color in the presence of varying predesigned colors;

In one embodiment, wherein the color calibration region includes a plurality of calibration regions, each of which has a different calibration color, or the calibration region includes control samples that have predesignated concentration of indicia of the sample; obtaining both of the color image of the test region containing the sample and the calibration region using a mobile device including an optional light source and/or optional an image detector; processing the images of nucleic acid amplification on the mobile device or sending out the information to a cloud service;

In one embodiment, it includes an optional time stamping, determining selected quantitative indicia of the sample and storing the determined value for future access; location stamping the determined selected quantitative indicia of the sample and storing the determined value for future access; storing the time and/or location data in at least one of a readable file in the mobile device, an external readable file, and in a cloud file; determining a temporal and/or a location trend of a plurality of the determined selected quantitative indicia of the sample; correlating the determined selected quantitative indicia of the sample to a related selected metric and displaying a value of the related selected metric on the mobile device;

In one embodiment, wherein the sample includes but not limited to sweat, saliva, blood, tears, urine, other bodily fluids, tissue, food, produce, soil or any substance that may contain nucleic acids, DNA and/or RNA from one or combinations of organisms and viruses: animals, plants, microorganisms;

The heating material is suitably in thermal communication with the reaction chamber of a test platform, or any combination thereof.

Heating materials that are chemically reactive are considered especially suitable. Such materials include magnesium-iron alloy, calcium oxide, sodium acetate, potassium permanganate (reactive with glycerol), and the like. Or heating materials may be chemically inert materials such as mineral oil or water.

In one embodiment, the mobile device accessory comprises a heating source. The heating source comprises a heating element, a thermal storage medium in thermal communication with the heat element, the thermal storage medium comprising a phase changing material (PCM). The heat source and thermal storage medium configured to maintain the temperature of a reaction chamber adjacent to the heat source.

In one exemplary embodiment, the mobile device accessory comprises a heating material that undergoes an exothermic reaction upon contacting with a fluid; a thermal storage medium in thermal communication with the heat source, the thermal storage medium comprising a phase changing material (PCM).

The system may include a reaction chamber for biochemical reaction. Such reaction chambers may be adapted for nucleic acid amplification or sample preparation. In one of embodiments, the reaction chamber may contain one or more pre-stored (e.g., dried) reagents within or reagent sealed with wax.

In one embodiment, the heat source comprises an electrical thermostat device.

In one embodiment, the heat source includes but not limited to an electric thermos or electric kettle.

In one embodiment, the heat source comprises an electric heating element to maintain temperatures for various stages of nucleic acid amplification reaction or sample preparation.

The heating element has multiple temperature settings.

In one embodiment, a PTC or NTC heating element is the heat source and used to maintain nucleic acid amplification reaction.

In one embodiment, a fluid such as water is thermal conductive medium or heating material to facilitate heat communication between reaction chamber of nucleic acid amplification reaction and a heat source.

In one non-limiting embodiment, the heat source and thermal storage medium may be configured to maintain the temperature of a sample in a reaction chamber adjacent to the heat source at a temperature in the range of from about 25 deg.C. to about 100 deg.C. for a period of time from about 1 second to about 120 minutes in an environment of ambient temperature ranging from 5 deg.C. to 50 deg.C.

In one embodiment, a cartridge or a kit comprises reagents for PCR or isothermal amplification reaction (Isothermal amplification of nucleic acids, Chem Rev, 115,12491 (2015)), DNA polymerase, DNA primers and/or control DNA and/or fluorescence dyes/and/or pH indicator and/or magnesium indicator. Or. The kit comprises lyophilized reagents and may be used by adding buffer or water. In one embodiment, the kit may include a wax bead for PCR reagents (U.S. Pat. No. 5,413,924) or a wax sealed PCR master mix.

A variety of fluidic elements may be present. Such elements may be valves, pistons, membranes, cantilevers, and the like. In this way, the heating material, thermal storage medium or heat source may be configured so as to supply sufficient heat to the reaction chamber of a test platform.

In one embodiment, thermal storage medium used in the disclosed devices may include a wax, a thermoplastic, a salt hydrate, a fatty acid, a fatty acid ester, or any combination thereof. Paraffin wax is considered a particularly suitable thermal storage medium that undergoes a phase change at a particular temperature. Such materials permit the construction of devices that controllably maintain a particular temperature, which temperature is regulated by the phase change temperature (e.g., melting) of the thermal storage medium.

In one embodiment, water is added into a heating material of a heat source containing the calcium oxide powder, which heats up a phase change material. The heat source's temperature is regulated and rendered independent of ambient temperatures with the aid of a phase change material.

In one of embodiment, PCM is heat up by a physical process, thereby it is possible to reuse said heating element.

In one embodiment, a chemically inert liquid or vapor retarders is added to water to slow down temperature decreasing.

In other embodiments, a conductive material (e.g., metal) places the heating material in thermal communication with the reaction chamber of a test platform or other components of the system. The devices also include one or more manually-operated elements that allow the user to place various components of the device into fluid or thermal contact with one another.

Also provided are methods of processing a sample. These methods suitably include contacting a chemically reactive heat source with a fluid so as to generate heat; the chemically reactive heat source being in thermal communication with a thermal (e.g., heat) storage medium.

As discussed elsewhere herein, the heat source or heating material, once activated or heated up, serves to supply heat to the reaction chamber of a test platform or other elements. In this manner, a system can be constructed that is capable of performing a reaction or other process that requires heat, while the PCM store and regulate the release of heat.

In some embodiments, an amount of fluid (e.g., water, oil, wax) is packaged with the device or added by user with specified amount of fluid so that the fluid is available to the device at the time of activation or heating up.

In one embodiment, it comprises at least two heating sources, and each has different temperature from other. The different temperature of heating sources may facilitate the nucleic acid amplification reaction at different stages or reactions at sample preparation. For a non-limited example, contacting a heat source at around 95 deg. C., the product DNAs separate from the target DNAs while contacting another heat source at around 72 deg. C., polymerase synthesize DNA products.

In one embodiment, the system comprises a motor or actuator. Through a linkage of a motor or actuator, the system translocates the reaction chamber at a test platform relatively from a heat source at one temperature to other heat source at a different temperature, and the reaction chamber contacts with each heat source for a predesigned period of time in order to complete a reaction stage.

By translocation of the reaction chamber between different heating sources relatively, the reaction chamber may have thermal communications with a particular heat source. Thereby, the temperature of nucleic acid amplification reaction (i.e. PCR or isothermal amplification reaction) also switches between different temperatures when the reaction chamber is moved to the proximity of a heat source. Thereby, the reaction stages can be controlled by shuttling the reaction chamber to the proximity of a heat source.

In one embodiment, a cycle of PCR can be achieved by translocating the reaction chamber adjacent to different heat sources with temperatures suitable for denaturation of DNA, annealing of DNA and synthesis of DNA.

In one embodiment, translocation of a reaction chamber relatively to heat sources is for sample preparation such as cell lysis or reverse transcription of RNA.

In one embodiment, steps of sample preparation and nucleic acid amplification are integrated by moving reaction chamber relatively over different heat sources with different temperatures for each particular step or reaction.

In one embodiment, a reaction chamber contacts a cell sample and reagents while the reaction chamber also contacts with a heat source with a specific temperature for thermal communication. Therefore, the cell sample and reagent would reach or close to thermal equilibrium with the heat source. The cell sample is then lysed at the specific temperature that is suitable for such lysis reaction. Once lysis reaction is finished, the reaction chamber is moved relatively for contacting a heat source with a temperature suitable for a particular stage of nucleic acid amplification reaction. One example of the specific temperature is DNA denaturation temperature.

In one embodiment, it comprises the three heat sources, and each for primer annealing, DNA extension and DNA denaturation. A mechanic mean moves the reaction chamber among the three heating elements. Thereby, the reaction chamber goes from different temperatures while moving among the three heat sources for a cycle PCR. The cycle repeats till a certain number of cycles is reached. The result of PCR is determined by the color change of reaction product via said mobile device.

One of embodiments is a portable, modular, point-of-collection, colorimetric-based system for nucleic acid amplification reaction.

In one embodiment, wherein the accessory include a heat source and the accessory is adapted to receive a modular, colorimetric test platform in a manner that allows exposure of the test region and color calibration region of a test platform to a light source;

In one embodiment, wherein said light source is an external light source such as ambient light or a LED light; and an executable software resident in the mobile device that, in operation, performs the following steps: acquires an image of at least a portion of the test region of a test platform; stores the image as an RGBA/YUV byte array; splits the image into a test image and/or a calibration image; for the calibration image: extracts a calibration array of pixels; determines a median or average RGBA/YUV color coordinate for the calibration array of pixels; maps the median or average RGBA color coordinates of the calibration array of pixels to the designated value; and for the test image: extracts a test array of pixels; determines a median or average RGBA color coordinate of the test array of pixels; maps the median or average RGBA color coordinate from the test array of pixels with the same mapping function used for calibration image; and determines a quantitative value of the selected indicia of the a sample to be measured by using a threshold for the mapped values obtained from test and calibration image. The mapped value is to associate the amount of nucleic acid in a reaction. The association of nucleic acid quantitative or qualitative amount is though a mapping function.

In one embodiment, the mapped value is a hue value from RGBA color coordinate of the test array of pixels or calibration array of pixels, respectively.

In one embodiment, creating a mapping function is performing a root-polynomial regression of calibration RGBA coordinate over designated RGBA coordinate, and then follows by the conversion of RGBA coordinate to Hue-Saturation-Intensity (HSI) space. Once a median or average RGBA color coordinate for the test array of pixels convert to the Hue value of HIS space by the mapping function, one can determine if the reaction is successful or not against a predesigned value.

In one embodiment, the process of mapping RGBA coordinates and determining the outcome of a reaction is through a machine learning process (Smartphone-based colorimetric detection via machine learning, Analyst, 142, 2434(2017)).

In one embodiment, said mapping function is an identity function.

In one embodiment, a color subtraction approach is used to determine the color change (Smartphone Modulated Colorimetric Reader with Color Subtraction, DOI: 10.1109/SENSORS43011.2019.8956565, (2019)).

In one embodiment, the light source is an external or internal flash source of the mobile device or ambient light source;

In one embodiment, the light source is an LED disposed in the mobile device accessory, further comprising a battery in the mobile device accessory to power the LED or the accessory is powered by electrical grid or a battery;

In one embodiment, wherein the mobile device accessory includes a light diffuser and/or a light-diffusing pathway so as to ensure a uniform and repeatable illumination of at least a desired region of the modular, colorimetric test platform; wherein the colorimetric reactive test platform includes a colorimetric reactive test region and a colorimetric reactive or non-colorimetric reactive calibration region;

In one embodiment, wherein the colorimetric reactive test region is a test region for colorimetric reactive nucleic acid amplification reaction, wherein the light diffuser is disposed on at least a portion of a surface of the test platform is in such a manner to provide diffuse illumination to a surface of the test platform; In one embodiment, wherein the non-colorimetric reactive calibration region comprises a glossy material.

One of the embodiments is a method for obtaining a point-of-collection, selected quantitative indicia of a sample on a test platform with a mobile device. Illustrative method steps include providing a modular, colorimetric reactive test platform having a test region and a calibration region; providing an sample to be tested on the test region of the modular, colorimetric test platform;

In one embodiment, the test platform may have a plurality of reaction chambers.

In the test platform, each reaction chamber contains pre-dry primer sets. The primer set in a reaction chamber may target to a genome sequence location of one or a plurality of organisms.

In one embodiment, nucleic acid extracted from at least one sample is dispensed to a test platform with plurality of reaction chambers.

In one embodiment, a primer set is designed by the procedure: select at least one interested organism or virus, and extract a coding sequences from the EMBL coding domain sequence database, clustered 96% sequence identity Use the sequences as target sequences, and select primers with close melting temperature and similar amplicon sizes. In one embodiment, the amplicon size is 90 nt to 150 nt. In one embodiment, the selection is through Primer3 (Untergasser A, Cutcutache I, Koressaar T, Ye J, Faircloth B C, Remm M, Rozen S G (2012) Primer3—new capabilities and interfaces. Nucleic Acids Research 40(15):e115; Koressaar T, Remm M Enhancements and modifications of primer design program Primer3 Bioinformatics 23(10):1289 (2007)).

The exemplary viruses are listed in Tables 1—which is derived from U.S. Pat. No. 10,815,536, issued Oct. 27, 2020, and entitled “Virome Capture Sequencing Platform, Method of Designing And Constructing and Methods of Using”)

TABLE 1
Virus Taxa Selected for primer Design

| | | | Parent |
|---|---|---|---|
| Name | tax_id | Parent Name | tax_id |
| Adenoviridae | 10508 | dsDNA viruses, | 35237 |
| | | no RNA stage | |
| Alloherpesviridae | 548682 | Herpesvirales | 548681 |
| Alphacoronavirus | 693996 | Coronavirinae | 693995 |
| Alphaherpesvirinae | 10293 | Herpesviridae | 10292 |
| Alphanodavirus | 143920 | Nodaviridae | 12283 |
| Alphapapillomavirus | 333750 | Papillomaviridae | 151340 |
| Alphapermutotetravirus | 1283211 | Permutotetraviridae | 1283210 |
| Alpharetrovirus | 153057 | Orthoretrovirinae | 327045 |
| Alphatorquevirus | 687331 | Anelloviridae | 687329 |
| Alphavirus | 11019 | Togaviridae | 11018 |
| Amdoparvovirus | 310911 | Parvovirinae | 40119 |
| Anelloviridae | 687329 | ssDNA viruses | 29258 |
| Aphthovirus | 12109 | Picornaviridae | 12058 |
| Aquabirnavirus | 39750 | Birnaviridae | 10993 |
| Aquamavirus | 1330065 | Picornaviridae | 12058 |
| Aquaparamyxovirus | 1232658 | Paramyxovirinae | 11159 |
| Aquareovirus | 10979 | Spinareovirinae | 689831 |
| Arenaviridae | 11617 | SSRNA negative - | 35301 |
| | | strand viruses | |
| Arenavirus | 11618 | Arenaviridae | 11617 |
| Arteriviridae | 76803 | Nidovirales | 76804 |
| Arterivirus | 11046 | Arteriviridae | 76803 |
| Asfarviridae | 137992 | dsDNA viruses, | 35237 |
| | | no RNA stage | |
| Asfivirus | 39743 | Asfarviridae | 137992 |
| Astroviridae | 39733 | ssRNA positive - | 35278 |
| | | strand viruses, | |
| | | no DNA stage | |
| Atadenovirus | 100953 | Adenoviridae | 10508 |
| Aurivirus | 1513230 | Malacoherpesviridae | 548685 |
| Avastrovirus | 249589 | Astroviridae | 39733 |
| Aveparvovirus | 1511864 | Parvovirinae | 40119 |
| Aviadenovirus | 10552 | Adenoviridae | 10508 |
| Avibimavirus | 39751 | Birnaviridae | 10993 |
| Avihepadnavirus | 10437 | Hepadnaviridae | 10404 |
| Avihepatovirus | 691955 | Picornaviridae | 12058 |
| Avipoxvirus | 10260 | Chordopoxvirinae | 10241 |
| Avisivirus | 1511771 | Picornaviridae | 12058 |
| Avulavirus | 260963 | Paramyxovirinae | 11159 |
| Bafinivirus | 694018 | Torovirinae | 694017 |
| Batrachovirus | 692605 | Alloherpesviridae | 548682 |
| Betacoronavirus | 694002 | Coronavirinae | 693995 |
| Betaherpesvirinae | 10357 | Herpesviridae | 10292 |
| Betanodavirus | 143919 | Nodaviridae | 12283 |
| Betapapillomavirus | 333922 | Papillomaviridae | 151340 |
| Betaretrovirus | 140052 | Orthoretrovirinae | 327045 |
| Betatorquevirus | 687332 | Anelloviridae | 687329 |
| Birnaviridae | 10993 | dsRNA viruses | 35325 |
| Blosnavirus | 564643 | Birnaviridae | 10993 |
| Bocaparvovirus | 1507401 | Parvovirinae | 40119 |
| Bornaviridae | 178830 | Mononegavirales | 11157 |
| Bornavirus | 186458 | Bornaviridae | 178830 |
| Bracorhabdovirus | 490109 | unclassified | 35303 |
| | | Rhabdoviridae | |
| Bunyaviridae | 11571 | SSRNA negative - | 35301 |
| | | strand viruses | |
| Caliciviridae | 11974 | SSRNA positive - | 35278 |
| | | strand viruses, | |
| | | no DNA stage | |
| Capripoxvirus | 10265 | Chordopoxvirinae | 10241 |
| Cardiovirus | 12103 | Picornaviridae | 12058 |
| Cervidpoxvirus | 573055 | Chordopoxvirinae | 10241 |
| Chipapillomavirus | 934800 | Papillomaviridae | 151340 |
| Chloriridovirus | 10491 | Iridoviridae | 10486 |
| Chordopoxvirinae | 10241 | Poxviridae | 10240 |
| Circoviridae | 39724 | ssDNA viruses | 29258 |
| Circovirus | 39725 | Circoviridae | 39724 |
| Coltivirus | 10911 | Spinareovirinae | 689831 |
| Copiparvovirus | 1511888 | Parvovirinae | 40119 |
| Coronaviridae | 11118 | Nidovirales | 76804 |
| Coronavirinae | 693995 | Coronaviridae | 11118 |
| Cosavirus | 586418 | Picornaviridae | 12058 |
| Crocodylidpoxvirus | 1285599 | Chordopoxvirinae | 10241 |
| Cuevavirus | 1513236 | Filoviridae | 11266 |
| Cyprinivirus | 692606 | Alloherpesviridae | 548682 |
| Cytomegalovirus | 10358 | Betaherpesvirinae | 10357 |
| Cytorhabdovirus | 11305 | Rhabdoviridae | 11270 |
| Deltacoronavirus | 1159901 | Coronavirinae | 693995 |
| Deltapapillomavirus | 325454 | Papillomaviridae | 151340 |
| Deltaretrovirus | 153136 | Orthoretrovirinae | 327045 |
| Deltatorquevirus | 687334 | Anelloviridae | 687329 |
| Deltavirus | 39759 | Viruses | 10239 |
| Dengue virus group | 11052 | Flavivirus | 11051 |
| Densovirinae | 40120 | Parvoviridae | 10780 |
| Dependoparvovirus | 10803 | Parvovirinae | 40119 |
| Dicipivirus | 1330067 | Picornaviridae | 12058 |
| Dinornavirus | 674976 | Alvernaviridae | 866787 |
| Dyodeltapapillomavirus | 936056 | Papillomaviridae | 151340 |
| Dyoepsilonpapillomavirus | 935646 | Papillomaviridae | 151340 |
| Dyoetapapillomavirus | 935641 | Papillomaviridae | 151340 |
| Dyoiotapapillomavirus | 934804 | Papillomaviridae | 151340 |
| Dyokappapapillomavirus | 1513238 | Papillomaviridae | 151340 |
| Dyolambdapapillomavirus | 1513239 | Papillomaviridae | 151340 |
| Dyomupapillomavirus | 1513240 | Papillomaviridae | 151340 |
| Dyonupapillomavirus | 1513241 | Papillomaviridae | 151340 |
| Dyoomikronpapillomavirus | 1513242 | Papillomaviridae | 151340 |
| Dyopipapillomavirus | 1513243 | Papillomaviridae | 151340 |
| Dyorhopapillomavirus | 1513244 | Papillomaviridae | 151340 |
| Dyosigmapapillomavirus | 1513245 | Papillomaviridae | 151340 |
| Dyothetapapillomavirus | 1052159 | Papillomaviridae | 151340 |
| Dyoxipapillomavirus | 1513246 | Papillomaviridae | 151340 |
| Dyozetapapillomavirus | 934803 | Papillomaviridae | 151340 |
| Ebolavirus | 186536 | Filoviridae | 11266 |
| Enterovirus | 12059 | Picornaviridae | 12058 |
| Entomopoxvirinae | 10284 | Poxviridae | 10240 |
| Ephemerovirus | 32613 | Rhabdoviridae | 11270 |
| Epsilonretrovirus | 153137 | Orthoretrovirinae | 327045 |
| Epsilontorquevirus | 687335 | Anelloviridae | 687329 |
| Equine lentivirus group | 11654 | Lentivirus | 11646 |
| Erbovirus | 194961 | Picornaviridae | 12058 |
| Erythroparvovirus | 40121 | Parvovirinae | 40119 |
| Etapapillomavirus | 325458 | Papillomaviridae | 151340 |
| Etatorquevirus | 687337 | Anelloviridae | 687329 |
| Ferlavirus | 1283308 | Paramyxovirinae | 11159 |
| Filoviridae | 11266 | Mononegavirales | 11157 |
| Flaviviridae | 11050 | SsRNA positive - | 35278 |
| | | strand viruses, | |
| | | no DNA stage | |
| Flavivirus | 11051 | Flaviviridae | 11050 |
| Gallivirus | 1511775 | Picornaviridae | 12058 |
| Gammacoronavirus | 694013 | Coronavirinae | 693995 |
| Gammaherpesvirinae | 10374 | Herpesviridae | 10292 |
| Gammapapillomavirus | 325455 | Papillomaviridae | 151340 |
| Gammaretrovirus | 153135 | Orthoretrovirinae | 327045 |
| Gammatorquevirus | 687333 | Anelloviridae | 687329 |
| Gyrovirus | 227307 | Circoviridae | 39724 |
| Hantavirus | 11598 | Bunyaviridae | 11571 |
| Henipavirus | 260964 | Paramyxovirinae | 11159 |
| Hepacivirus | 11102 | Flaviviridae | 11050 |
| Hepadnaviridae | 10404 | Retro - transcribing | 35268 |
| | | viruses | |
| Hepatovirus | 12091 | Picornaviridae | 12058 |
| Hepeviridae | 291484 | SsRNA positive - | 35278 |
| | | strand viruses, | |
| | | no DNA stage | |
| Hepevirus | 186677 | Hepeviridae | 291484 |
| Herpesvirales | 548681 | dsDNA viruses, | 35237 |
| | | no RNA stage | |
| Herpesviridae | 10292 | Herpesvirales | 548681 |
| Hunnivirus | 1431456 | Picornaviridae | 12058 |
| Ichtadenovirus | 691957 | Adenoviridae | 10508 |
| Ictalurivirus | 172653 | Alloherpesviridae | 548682 |
| Iltovirus | 180255 | Alphaherpesvirinae | 10293 |
| Influenzavirus D | 1511083 | unclassified | 35324 |
| | | Orthomyxoviridae | |
| Intracisternal A - particles | 11749 | unclassified | 35276 |
| | | Retroviridae | |
| Iotatorquevirus | 687339 | Anelloviridae | 687329 |
| Iridoviridae | 10486 | dsDNA viruses, | 35237 |
| | | no RNA stage | |
| Iridovirus | 10487 | Iridoviridae | 10486 |
| Isavirus | 324913 | Orthomyxoviridae | 11308 |
| Japanese encephalitis | 11071 | Flaviviru | 11051 |
| virus group | | | |
| Kappapapillomavirus | 325457 | Papillomaviridae | 151340 |
| Kappatorquevirus | 1218487 | Anelloviridae | 687329 |
| Kobuvirus | 194960 | Picornaviridae | 12058 |
| Kokobera virus group | 303179 | Flavivirus | 11051 |
| Lagovirus | 95339 | Caliciviridae | 11974 |
| Lambdapapillomavirus | 325462 | Papillomaviridae | 151340 |
| Lambdatorquevirus | 1218489 | Anelloviridae | 687329 |
| Lentivirus | 11646 | Orthoretrovirinae | 327045 |
| Leporipoxvirus | 10270 | Chordopoxvirinae | 10241 |
| Lymphocryptovirus | 10375 | Gammaherpesvirinae | 10374 |
| Lymphocystivirus | 10494 | Iridoviridae | 10486 |
| Lyssavirus | 11286 | Rhabdoviridae | 11270 |
| Macavirus | 548687 | Gammaherpesvirinae | 10374 |
| Malacoherpesviridae | 548685 | Herpesvirales | 548681 |
| Mamastrovirus | 249588 | Astroviridae | 39733 |
| Marburgvirus | 186537 | Filoviridae | 11266 |
| Mardivirus | 180252 | Alphaherpesvirinae | 10293 |
| Mastadenovirus | 10509 | Adenoviridae | 10508 |
| Megalocytivirus | 308906 | Iridoviridae | 10486 |
| Megrivirus | 1330069 | Picornaviridae | 12058 |
| Metapneumovirus | 162387 | Pneumovirinae | 11244 |
| Mischivirus | 1511778 | Picornaviridae | 12058 |
| Modoc virus group | 29260 | Flavivirus | 11051 |
| Molluscipoxvirus | 10278 | Chordopoxvirinae | 10241 |
| Mononegavirales | 11157 | SSRNA negative - | 35301 |
| | | strand viruses | |
| Morbillivirus | 11229 | Paramyxovirinae | 11159 |
| Mosavirus | 1481451 | Picornaviridae | 12058 |
| mosquito - borne viruses | 59562 | Flavivirus | 11051 |
| Mupapillomavirus | 334202 | Papillomaviridae | 151340 |
| Muromegalovirus | 10365 | Betaherpesvirinae | 10357 |
| Nairovirus | 11592 | Bunyaviridae | 11571 |
| Nebovirus | 696855 | Caliciviridae | 11974 |
| Negevirus | 1307798 | unclassified | 38173 |
| | | ssRNA positive | |
| | | strand viruses | |
| Nidovirales | 76804 | SsRNA positive - | 35278 |
| | | strand viruses, | |
| | | no DNA stage | |
| Nodaviridae | 12283 | SSRNA positive - | 35278 |
| | | strand viruses, | |
| | | no DNA stage | |
| Norovirus | 142786 | Caliciviridae | 11974 |
| Novirhabdovirus | 186778 | Rhabdoviridae | 11270 |
| Ntaya virus group | 29261 | Flavivirus | 11051 |
| Nucleorhabdovirus | 11306 | Rhabdoviridae | 11270 |
| Nupapillomavirus | 475861 | Papillomaviridae | 151340 |
| Nyamiviridae | 1513294 | Mononegavirales | 11157 |
| Nyavirus | 1513295 | Nyamiviridae | 1513294 |
| Omegapapillomavirus | 936061 | Papillomaviridae | 151340 |
| Orbivirus | 10892 | Sedoreovirinae | 689832 |
| Orthobunyavirus | 11572 | Bunyaviridae | 11571 |
| Orthohepadnavirus | 10405 | Hepadnaviridae | 10404 |
| Orthomyxoviridae | 11308 | SSRNA negative - | 35301 |
| | | strand viruses | |
| Orthopoxvirus | 10242 | Chordopoxvirinae | 10241 |
| Orthoreovirus | 10882 | Spinareovirinae | 689831 |
| Orthoretrovirinae | 327045 | Retroviridae | 11632 |
| Oscivirus | 1511780 | Picornaviridae | 12058 |
| Ostreavirus | 548686 | Malacoherpesviridae | 548685 |
| Papillomaviridae | 151340 | dsDNA viruses, | 35237 |
| | | no RNA stage | |
| Paramyxoviridae | 11158 | Mononegavirales | 11157 |
| Paramyxovirinae | 11159 | Paramyxoviridae | 11158 |
| Parapoxvirus | 10257 | Chordopoxvirinae | 10241 |
| Parechovirus | 138954 | Picornaviridae | 12058 |
| Parvoviridae | 10780 | SSDNA viruses | 29258 |
| Parvovirinae | 40119 | Parvoviridae | 10780 |
| Pasivirus | 1511782 | Picornaviridae | 12058 |
| Passerivirus | 1511802 | Picornaviridae | 12058 |
| Pegivirus | 1307799 | Flaviviridae | 11050 |
| Percavirus | 548688 | Gammaherpesvirinae | 10374 |
| Perhabdovirus | 1298653 | Rhabdoviridae | 11270 |
| Pestivirus | 11095 | Flaviviridae | 11050 |
| Phipapillomavirus | 934802 | Papillomaviridae | 151340 |
| Phlebovirus | 11584 | Bunyaviridae | 11571 |
| Picobirnaviridae | 585893 | dsRNA viruses | 35325 |
| Picobirnavirus | 104394 | Picobirnaviridae | 585893 |
| Picornavirales | 464095 | ssRNA positive - | 35278 |
| | | strand viruses, | |
| | | no DNA stage | |
| Picornaviridae | 12058 | Picomavirales | 464095 |
| Pipapillomavirus | 334211 | Papillomaviridae | 151340 |
| Pneumovirinae | 11244 | Paramyxoviridae | 11158 |
| Pneumovirus | 11245 | Pneumovirinae | 11244 |
| Polyomaviridae | 151341 | dsDNA viruses, | 35237 |
| | | no RNA stage | |
| Polyomavirus | 10624 | Polyomaviridae | 151341 |
| Poxviridae | 10240 | dsDNA viruses, | 35237 |
| | | no RNA stage | |
| Proboscivirus | 548689 | Betaherpesvirinae | 10357 |
| Protoparvovirus | 1506574 | Parvovirinae | 40119 |
| Psipapillomavirus | 935650 | Papillomaviridae | 151340 |
| Quadrivirus | 1299297 | Quadriviridae | 1299296 |
| Quaranjavirus | 1299308 | Orthomyxoviridae | 11308 |
| Ranavirus | 10492 | Iridoviridae | 10486 |
| Recovirus | 873551 | Caliciviridae | 11974 |
| Reoviridae | 10880 | dsRNA viruses | 35325 |
| Respirovirus | 186938 | Paramyxovirinae | 11159 |
| Retroviridae | 11632 | Retro - transcribing | 35268 |
| | | viruses | |
| Rhabdoviridae | 11270 | Mononegavirales | 11157 |
| Rhadinovirus | 10379 | Gammaherpesvirinae | 10374 |
| Rhopapillomavirus | 936057 | Papillomaviridae | 151340 |
| Rio Bravo virus group | 29262 | Flavivirus | 11051 |
| Rosavirus | 1511804 | Picomaviridae | 12058 |
| Roseolovirus | 40272 | Betaherpesvirinae | 10357 |
| Rotavirus | 10912 | Sedoreovirinae | 689832 |
| Rubivirus | 11040 | Togaviridae | 11018 |
| Rubulavirus | 39744 | Paramyxovirinae | 11159 |
| Salivirus | 688449 | Picornaviridae | 12058 |
| Salmonivirus | 692607 | Alloherpesviridae | 548682 |
| Sapelovirus | 686982 | Picornaviridae | 12058 |
| Sapovirus | 95341 | Caliciviridae | 11974 |
| Scutavirus | 1232637 | Alphaherpesvirinae | 10293 |
| Seaborne tick - bome | 29264 | Flavivirus | 11051 |
| virus group | | | |
| Seadornavirus | 208294 | Sedoreovirinae | 689832 |
| Sedoreovirinae | 689832 | Reoviridae | 10880 |
| Senecavirus | 586425 | Picornaviridae | 12058 |
| Siadenovirus | 129876 | Adenoviridae | 10508 |
| Sigmapapillomavirus | 935635 | Papillomaviridae | 151340 |
| Sigmavirus | 1308858 | Rhabdoviridae | 11270 |
| Simplexvirus | 10294 | Alphaherpesvirinae | 10293 |
| Spinareovirinae | 689831 | Reoviridae | 10880 |
| Sprivivirus | 1513299 | Rhabdoviridae | 11270 |
| Spumaretrovirinae | 327046 | Retroviridae | 11632 |
| Spumavirus | 11640 | Spumaretrovirinae | 327046 |
| Suipoxvirus | 10275 | Chordopoxvirinae | 10241 |
| Taupapillomavirus | 934799 | Papillomaviridae | 151340 |
| Teschovirus | 118139 | Picornaviridae | 12058 |
| Tetraparvovirus | 1511911 | Parvovirinae | 40119 |
| Thetapapillomavirus | 334213 | Papillomaviridae | 151340 |
| Thetatorquevirus | 687338 | Anelloviridae | 687329 |
| Thogotovirus | 35323 | Orthomyxoviridae | 11308 |
| Tibrovirus | 1299306 | Rhabdoviridae | 11270 |
| tick - borne encephalitis | 29263 | Flavivirus | 11051 |
| virus group | | | |
| Togaviridae | 11018 | ssRNA positive - | 35278 |
| | | strand viruses, | |
| | | no DNA stage | |
| Torovirinae | 694017 | Coronaviridae | 11118 |
| Torovirus | 11155 | Torovirinae | 694017 |
| Tremovirus | 689759 | Picornaviridae | 12058 |
| Tupavirus | 1513300 | Rhabdoviridae | 11270 |
| Upsilonpapillomavirus | 936058 | Papillomaviridae | 151340 |
| Varicellovirus | 10319 | Alphaherpesvirinae | 10293 |
| Vesiculovirus | 11271 | Rhabdoviridae | 11270 |
| Vesivirus | 95337 | Caliciviridae | 11974 |
| Yatapoxvirus | 10282 | Chordopoxvirinae | 10241 |
| Yellow fever virus group | 40005 | Flavivirus | 11051 |
| Zetapapillomavirus | 333918 | Papillomaviridae | 151340 |
| Zetatorquevirus | 687336 | Anelloviridae | 687329 |

In one embodiment, the meting temperature for primer is 55 deg. C. to 72 deg. C.

In one embodiment, choose the sequences of at least one interested organism or virus from databased which are complete sequences and have high coverage. The complete sequences are aligned using Cluster-Omega with default primers. The sequences are then removed excessive misaligned gaps for better identifying conserved polymorphic sites. Use trimAl tool to trim multiple sequence alignments (MSAs) as taught in (Design and in silico validation of polymerase chain reaction primers to detect severe actute respiratory syndrome coronavirus 2, Scientific Reports, 11,12565 (2021)). The sequences are subject to for primer design software such as MN908947 was used as a reference.

In one embodiment, the consensus-degenerate primers are designed and optimized as taught by (CODEHOP-mediated PCR—A powerful technique for the identification and characterization of viral genomes, Virology Journal 2: 20 (2005))

### BRIEF DESCRIPTION OF DRAWING

FIG. 1 illustrates a portable nucleic acid amplification system;

FIG. 2 illustrates a carrier which has four wheels and powered by a battery as a device for nucleic acid amplification;

FIG. 3 illustrates a receptacle. The receptacle can accommodate plurality of nucleic acid reactions, and has a thin and flat bottom surface as well;

FIG. 4 illustrate a chemically powered heat source comprises of a thermo, a chemically activated heating material, a aluminum foil cup, oil with low evaporation rate, water, and lid for the thermo;

FIG. 5 illustrates a receptacle accommodates plurality of capillaries, which can be a mean for transfer reagents or samples. Or the capillaries can serve as reaction chambers;

FIG. 6 illustrates the steps of a method for obtaining a point-of-collection, selected quantitative indicia of a sample on a test platform, according to an embodiment of the invention;

FIG. 7 is a high-level flow chart expressing the steps of a method for measuring a target of a nucleic acid amplification reaction in a biological sample using a mobile device according to an embodiment of the invention;

FIG. 8 is a flow chart of an embodiment method in terms of operational steps, procedures, reaction stages;

FIG. 9 is a mobile device adapted with a nanopore sequencer for nucleic acid sequencing;

FIG. 10 is a histogram of Hue values obtained from control and treatment samples;

FIG. 11 illustrates a portable nucleic acid amplification system with one heat source; and

FIG. 12 illustrates: a system of nucleic acid amplification with a mobile device using a tourbillion as means for driving the reaction chamber and controlling temperature of reaction.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims.

It is indeed intended that the scope of the invention should be determined by proper interpretation and construction of the appended claims and their legal equivalents, as understood by those of skill in the art relying upon the disclosure in this specification and the attached drawings.

### DETAILED DESCRIPTION OF EXEMPLARY EMBODIMENTS

As used herein, the term “move relatively” means the translocation between two positions is a motion between two positions in a system.

As used herein, the term “mobile device” means a mobile apparatus (or handheld computer) that is capable of running a programmed application suitable for executing the embodied functionality. It is a computer small enough to hold and operate in the hand. While suitable traditional smart phones may include products such as, e.g., the iPhone, iPad (Apple, Inc.), Android-based devices, Windows, HarmonyOS-based device and other well known devices and associated operating systems, the term mobile device as discussed and embodied herein is intended to include any digital mobile device such as smartphones, tablets, phablets, smart watches, mobile computer, digital camera, smart glass and other current or future smartphone platforms having similar minimal functionality.

In this regard and for the sake of clarity, a laptop computer might be covered under the definitional use of the term mobile device; but not a computing device that could be made portable or mobile by an accompanying apparatus that might give it portability mobility. Thus, the term “mobile device will be used herein (including the claims) to mean devices as discussed within the paragraph above.

It should be understood that the term “adjacent” (and in the claims) does not require that the reaction chamber be in directly contact with the heat source.

As used here “driven” shall include any form of drive mechanism or facilities for inducing motion in embodiments. It includes a combination of motor or gears, and the source of driving energy can be one or a combination of electric or mechanic or chemical energy.

As used here, “arm” shall include a linkage that may include one or more arms or leg members, bearings, and one or more receptacles for holding or gripping reaction chambers.

The term “colorimetric test platform”, “colorimetric measurement”, or “colorimetric reactive” as may be used herein means at least a measurable color change from one color to a different color or a measurable change in intensity of a particular color, in the presence of nucleic amplification reaction or due to temperature change of labels or reactions.

The term “suitable” as may be used herein (and in the claims) means having the qualities that are correct, needed, or appropriate for something, especially as a person skilled in the art would understand.

The term “about” as may be used herein means the amount of the specified quantity plus/minus a fractional amount thereof that a person skilled in the art would recognize as typical and reasonable for that particular quantity or measurement.

The term “test kits” or “kits” or “test platform” refers to a test platform; or a combination of the reagents required for nucleic acid amplification, a cartridge, a receptacle or reaction chambers for holding or storing said reagents or reaction.

Practical examples of embodied test platforms or test kit include, but are not limited to, various custom or commercially available test kits for nucleic acid amplification.

The term “light source” refers to ambient light source or light emitted by LED or light bulb or laser with a range of spectrum from 180 nm to 1064 nm.

The term “accessory” or “mobile device accessory” refers to a component of the system and the component is releasably coupled to the mobile device.

The term “indicia” refers to any physical quantity associated with the color coordinate. The physical quantities include but not limits to pyrophosphate concentration, proton concentration of the reaction, free magnesium ion concentration and amplified nucleic acid concentration, dye concentration or any reactants associate with amplified nucleic acid concentration.

The term “sample” refers to anything containing amplified nucleic acid and/or nucleic acids obtained from a sample of test.

The term “temperature label” is a material that change its color when the temperature of its contact changes.

The term “reactive test region” refers to a region of reaction chamber or a test platform, wherein nucleic acid amplification reaction is hold.

The term “temperature label” is a material that change its color when the temperature of its contact changes.

The term “reactive test region” refers to one or more of areas: a region of a kit, a cartridge, a reaction chamber, a receptacle, a test platform, wherein nucleic acid amplification reaction is hold.

The term “suction device” refers to a bulb or pump that can suck air or liquid from a capillary or a reaction chamber.

The DNA/RNA is extracted by the other component of the system from any fluid of a sample.

The other component is a nucleic acid extraction kit/module and/or an external device.

In one embodiment, a sample is collected and nucleic acid of samples is further processed in a reaction vessel.

In one embodiment, the reaction vessel is a reaction chamber.

In one of embodiments, the DNA/RNA for nucleic amplification reaction is introduced to a reaction chamber by a sample/reagent dispensing accessory.

In one embodiment, the sample/reagent dispensing accessory is one or more capillaries.

In one of embodiments, the test platform comprises a reactive test region or a reaction chamber, wherein the nucleic acid amplification occurs, and the adjacent heat source has heat communication with the reactive test region.

In one of embodiment, the reactive test region is a receptacle that holds at least one sample and all reagents required for nucleic acid amplification reaction, wherein the reactive test region is of interest area of colorimetric detection.

In one of embodiments, at least one accessory dispenses required reagents, enzymes, nucleotides, primers and samples into the reactive test region.

In one embodiment, a kit comprises nucleic acid amplification reagents for PCR or isothermal amplification reaction.

In one embodiment, the PCR is a convective polymerase chain reaction.

In one embodiment, nucleic acid amplification reagents includes but not limited to a combination of DNA polymerase and/or reverse transcriptase, nucleotide, reaction buffers, and/or nucleic acid primers for target nucleic acid fragments, and/or control nucleic acid; and sample preparation reagent may include a combination of cell lysis reagents and/or nucleic acid purification reagents

In one embodiment, the photo images of nucleic acid amplification reaction of a sample could be processed by the software installed on a mobile device. Therefore, the software identifies if a sample contains target nucleic acid sequences by analyzing the images of reaction through its color coordinate.

In one embodiment, the color coordinates from an image of test region is corrected against the color coordinate from the calibration region on the same image. Thereby the color difference from images taken by different mobile devices for a particular sample is corrected to a suitable range for colorimetric measurements.

In one embodiment, a lateral flow assay is performed with the amplified nucleic acid as taught in (Rapid One-Pot Detection of SARS-CoV-2 Based on a Lateral Flow Assay in Clinical Samples, Anal Chem. 93(7)3325 (2021)). The change of color lines on the lateral flow device is further determined by a colorimetric method via using a mobile device for the presence of an interested target.

A temperature label is a material changing its color with temperature. The change in colors is determined by the mobile device via the image of a temperature label. Therefore, the color change of the temperature label is used for monitoring the temperature of a system.

In one embodiment, the colorimetric-based method mentioned above is used with temperature label to determine the temperature of a system.

In one embodiment, the system comprises reagents for nucleic acid sequence amplification, a heat source, a PCM, a temperature label.

In one embodiment, the system comprises a kit for target nucleic acid sequence amplification, a mobile device, a heat source, a PCM, a temperature label and a tag.

In one embodiment, a tag may be taken into an image for analysis and/or registration of a test; wherein the image of tag is a QR code or 2D barcode.

In one embodiment, a tag contains information about the kit or samples or/and users including but not limited to the primers, reactants, enzymes, nucleotides, dye molecules, samples, user information, reagent or/and software version, geographic information, credential information.

In one embodiment, a tag can associate the mobile device with a cloud service.

In one embodiment, a tag can associate the nucleic acid amplification results and a cloud service.

In one embodiment, a tag can associate the geographic location where nucleic acid amplification performed or the location of said mobile device.

In one embodiment, a test platform comprises at least two reaction chambers. Each reaction on the test reaction chamber is associated with a unique tag. The tag is used to associate a reaction with a sample identity and/or amplification primer sets and/or geometry location and/or a time stamp.

In one embodiment, the test platform may be contained in a container, which has at least one side as being transparent to allow the detection of color change for image acquisition.

Furthermore, the software of system associates an information platform which not only identifies the samples or gene expression levels of samples but also provides further information for downstream treatment or management.

In one embodiment, the results of nucleic acid amplification and geographic location information are sent to cloud and the cloud provides recommendation for a user to take action based on the result or analysis.

In one embodiment, each reaction is collected in a different reactive test region of a container.

The container or each reaction region associates with a tag. A tag is used to further associate a reaction with a sample or amplification primer sets by the software, which provides convenience for user to operate sample preparation and record registration.

In one embodiment, the software is used to monitor the reaction conditions of nucleic acid. The conditions include temperature, amount of synthesized DNA, signal intensities with various temperatures or stages.

In one embodiment, the software can communicate with a heat source for temperature setting with a wire or wirelessly.

In one embodiment, the heating source can be an electric thermostat container.

A statistics method is performed to determine the likelihood of true positive result or true negative result.

In one embodiment, there are three or more than three samples as control samples while there are three or more than three samples as treatment samples.

In one embodiment, a t-test or ANOVA is performed to determine the confidence level of true positive or true negative result for samples.

In one embodiment, a p value of is provided to determine the significance level.

In one embodiment, at least one statistic methods is implemented in the mobile device of the system or on a cloud service which mobile device links to.

In one embodiment, the camera of a mobile device is used to directly collect images of reactive test region for determining nuclei acid amplification results. In the embodiment, the mobile device serves as a colorimeter by itself.

In one embodiment, a temperature label can associate with a heat source or a reactive test region, and the temperature label changes color when the temperature of heat source or of reactive test region changes. Thereby, the temperature of a heat source or reactive test region is monitored via images taken by a mobile device. The mobile device may have software installed, and the software can process the images for the color coordinates and determine the temperature of the heat source or reactive test region.

In one embodiment, quantification of amplification is by counting the sequence reads generated from a nanopore sequencer.

In one embodiment, nucleic acid amplification reaction agents include but not limited to a primer set for nucleic acid amplification reaction, DNA polymerase, nucleotide, reaction buffer.

In term of structure, one of the differences of invention from others is an enclosed house for current invention is optional.

In term of structure, one of the differences of invention from others is the system comprises a heat conductive reaction chamber which allow measuring indica of samples with a colorimetric method with a mobile device, and easy to scale up the number of reactions in a manner of point-of-collection.

In term of structure, one of the differences of invention from others is using electricity to power a heat source for nucleic acid amplification reaction or drive a reaction chamber is optional. Thereby the invention may be used in a resource limited area.

In term of structure, one of the differences of invention from others is said system comprises a mean for translocating a test platform or reaction chamber relatively to various position of a system for thermal communication with heat source or taking image with detection module of a mobile device or collection of nucleic acid amplification product.

In term of structure, one of the differences of invention from others is the detection module of a mobile device may be a nanopore DNA sequencer and/or an image sensor for sequencing or detection of amplified nucleic acid.

The present invention is directed to provide system and method of nucleic acid amplification in point-of-collection. The system comprises a heat source for facilitating the nucleic acid amplification reaction and a mobile device for measurement of nucleic acid amplification reaction. The measurement may include use of an image sensor for image acquisition and analysis of images or processing the sequencing data from the amplified nucleic acid produced by the system. The software is a method, and used to quantify amplified nucleic acid according to color change on an image taken or process the sequence data.

Because a thermal cycler requires a bulky system to conduct heat exchange when a large number of samples are required to process at the same time, it usually is difficult to handle more than 400 samples in a point-of-collection manner. In addition, it usually requires different temperatures for nucleic acid amplification and sample preparation or other biochemical reactions. Furthermore, detection of the result of target nucleic acid amplification during reaction or right after complete of reaction is favorable with a simple method such as colorimetric method or nucleic acid sequencing.

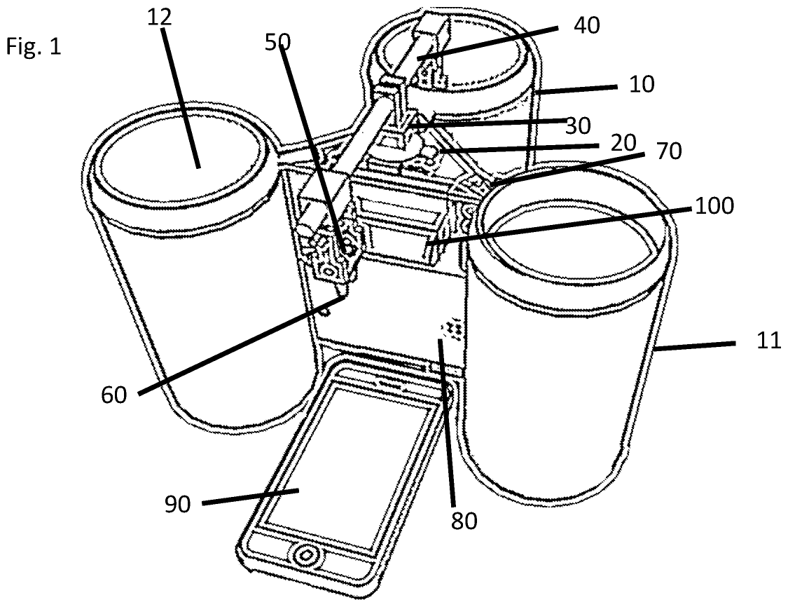

In FIG. 1, the exemplary embodiment shows three electric thermos10 with three different temperatures as three heat sources. Each has different temperature when filled with water. A test platform 20 is supported by the three thermos. On the platform, there is a step motor 30 connected to an arm 40. The arm hangs up a receptacle 50. And the receptacle accommodates a tube 60. The motor is powered by a battery 70. The position and duration of the arm is controlled by a programmed Arduino UNO R3 and UNL2003 board 80. By rotating the arm, the tube may immerse into each different thermos for each particular period of time. Since each thermo may have different temperatures corresponding to different stage of PCR reaction, immersing the tubes into different thermos may change the temperature of nucleic acid amplification, and cause the reaction to enter into different stages: denaturation of DNA, annealing DNA and DNA synthesis. Thereby, a PCR cycle can be complete by shuttling the tubes between the thermos.

Once the amplification reaction reaches the predesigned cycle number, the arm can move to the position just right above a cell phone 90. The software can take an image by a user or automatically take the pictures. A LED light source 100 may be used. It depends on which dye molecule is used to detect DNA. In one embodiment, the cell phone takes an image when each time the arm moves over the cell phone. Thereby, one may be able to monitor the amount of nucleic acid amplified over time.

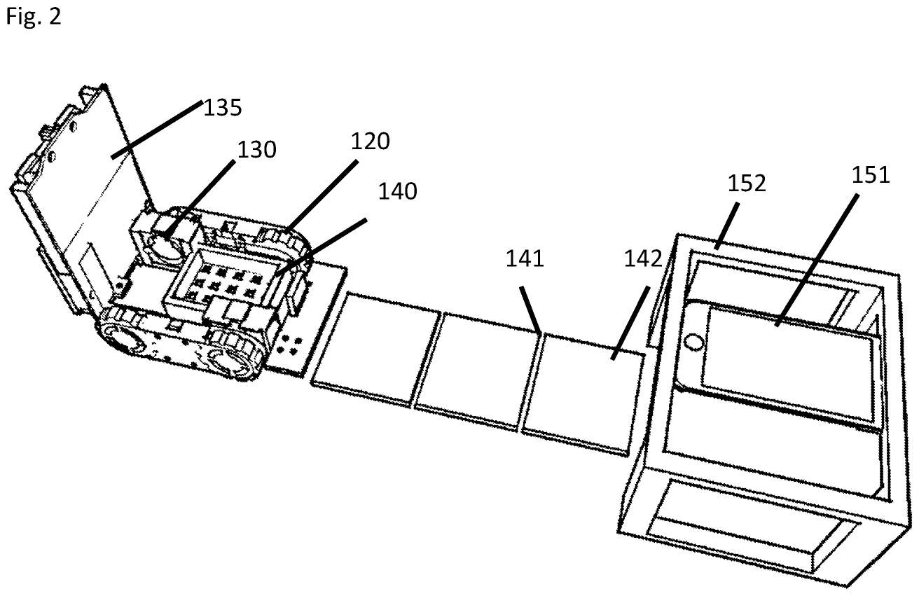

In FIG. 2, the embodiment shows a carrier has a receptacle 140 and wheels 120 driven by step motors 130, the receptacle has a flat bottom surface. The reaction chambers on the receptacle may contact with a hot plate with a preset temperature. The reaction chambers have openings on top and allow dispensing of reagents and samples. The reagent may contain liquid wax to seal the reaction chamber or prevent the evaporation of buffer from the reaction chambers. The carrier can move forward and backward over the hot plates with different temperatures such as 95 deg. C., 68 deg. C. The carrier can move over a hot plate with 95 deg. C. 141 for cell lysis with a specific time. And then, the carrier can further move to the hot plate with 68 deg. C. 142 for LAMP with other specific period. Finally, the carrier moves forward to facilitate imaging taken by a mobile device 151 on a holder 152. The carrier is controlled by an Arduino Uno R3 board 135.

The carrier can be used for PCR as well as isothermal nucleic acid amplification reaction. Samples and reagents may be dispensed to reaction chambers on the receptacle. The sample preparation may be performed at 95 deg. C. for DNA denaturation. The carrier may further move forward to a hot plate with 55 deg. C. for primer annealing and then move forward to a template with 72 deg. C. for DNA synthesis. Thereby, a PCR cycle can be complete via movement along three hot plates. Finally, the carrier moves to the position, which allows taking an image by a mobile device. The software installed on the mobile device can extract the RGB values from an image of reaction chambers and color calibration regions, mapping the RGB values to an associated hue value from HSI space. The hue vale may associate with a DNA test result or concentration.

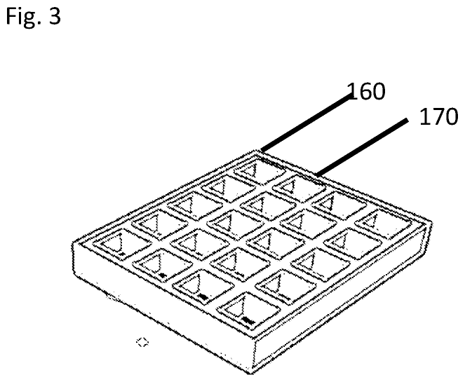

In FIG. 3, the embodiment schematically illustrates an exemplary embodiment of receptacle for a large number of nucleic acid amplification reactions. The receptacle 160 has plurality of reaction chambers 170 with opening on the top. The dimension of each reaction chamber is around 7 mm×7 mm×5 mm with a 5 mm thick of wall. For a silicon rubber heater with 100 cm×60 cm, it can easily accommodate several thousand of reaction chambers or reactions. The lyophilized plurality of primers sets is allocated into each reaction chamber respectively. Each of primer set may target a genome location of one or more organisms or viruses. The genome locations may be conservative or specific regions of genomes for interested organisms or viruses. An example of organisms or viruses for the primer sets is in the table 1.

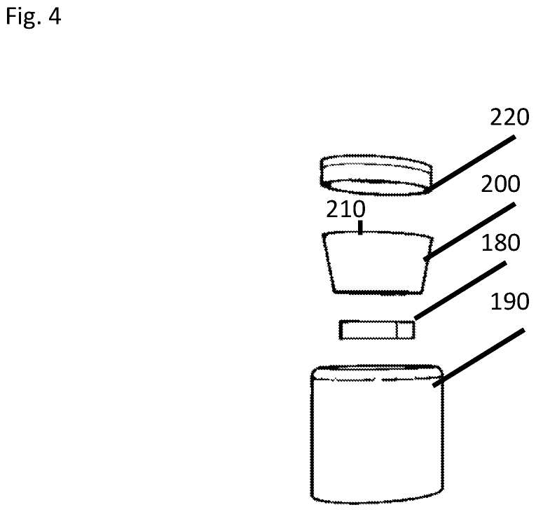

In FIG. 4, the embodiment shows an exemplary embodiment of chemically activated heat source. The heater 180 is inside the thermo 190. Above the heater is a foil cup 200 filled with water, and has oil or was on its top 210 to prevent quickly temperature drop. Oil with low evaporation rate at 95 deg. C., chemically inert with water and has lower density than water is suitable. Once activating the heater with adding water, the lid 220 of thermo is close till the predesigned temperature is reached, which can be observed by the temperature labels. One of examples of heaters is assortment of magnesium, iron and salt. Each heat source can have different temperature by the amount of heater and water added.

Thereby, the chemically activated heat source for a nucleic acid amplification reaction doesn't require electricity. The water in the foil cup may have thermal communication with a reaction chamber on a test platform.

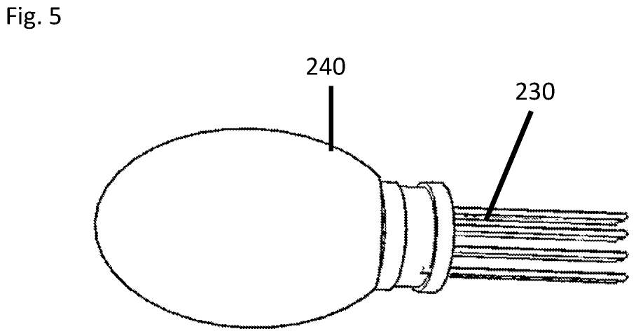

In FIG. 5, the embodiment schematically illustrates a capillary can be used as reaction chambers or a mean to transfer samples and reagents. One or more capillaries are immobilized on a receptacle, which is connected to a rubber bulb. The capillaries drawn a sample from a reservoir with a sample by capillary action and dispense the samples to receptacles which may contain reagents and buffer for nucleic acid amplification. After proper mixing of reagents and samples, the capillary can further draw the samples and reagents. The bottom end of a capillary may be fixed with fast-acting adhesive such as cyanoacrylate. The top of capillary may be sealed with wax. Thereby, the capillary may serve as a reaction chamber. In one application, saliva may be collected from an animal or person, the saliva may mix with cell lysis buffer in a reservoir and the reservoir is contacted with a heat source to maintain its temperature at 55 deg. C. to allow the cell lysis reaction to proceed. Once the reaction is complete, the capillaries may draw the nucleic acid from the reservoir and dispense into reaction chambers for nucleic acid amplification reactions and colorimetric measurement. Thereby, each capillary may correspond to a particular reaction chamber in the receptacle. The particular reaction chamber may contain a particular primer set which target a particular genome location of an animal or human.

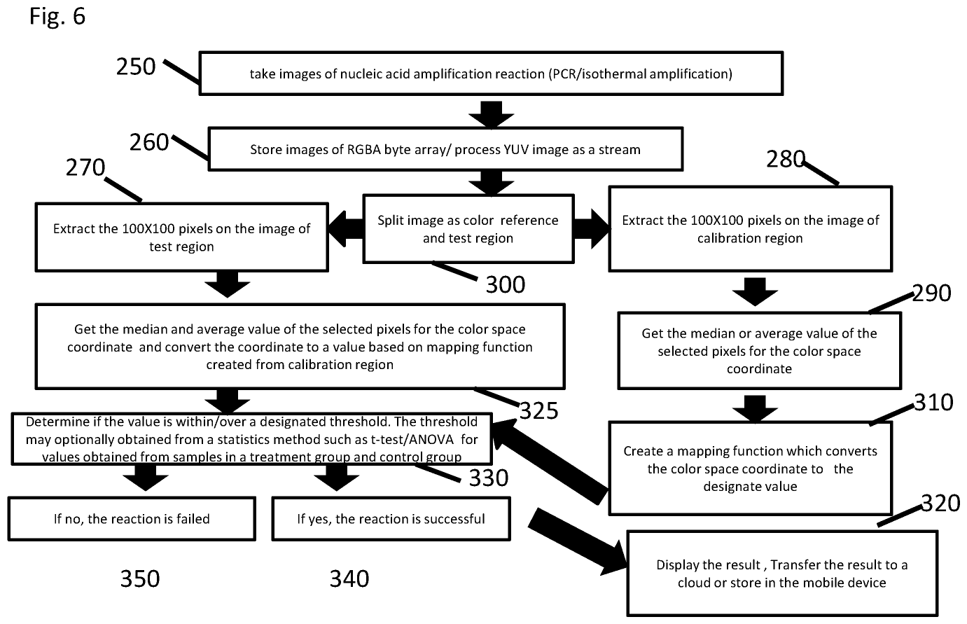

In FIG. 6, the embodiment schematically illustrates the steps for extract and analysis of an image from a nucleic acid reaction. It starts with collecting image of a nucleic acid amplification reaction 250. RGBA/YUV values are extracted from image 260, and then the values of image are split into test region and calibration region 300. Extract 100×100 pixels from the calibration region 280. From the median/average values obtained from calibration region 290, a mapping function which can convert measured value to a predesigned value 310. Extract 100×100 pixels from test region 270. The mapping function is then used to convert the median/average of measure values from test region 325 to a value against a designated threshold 330. If the value is over a threshold, the reaction successes 350 otherwise fails 340. The result eventually would be display and stored on a mobile device or transfer the data to a cloud 320.

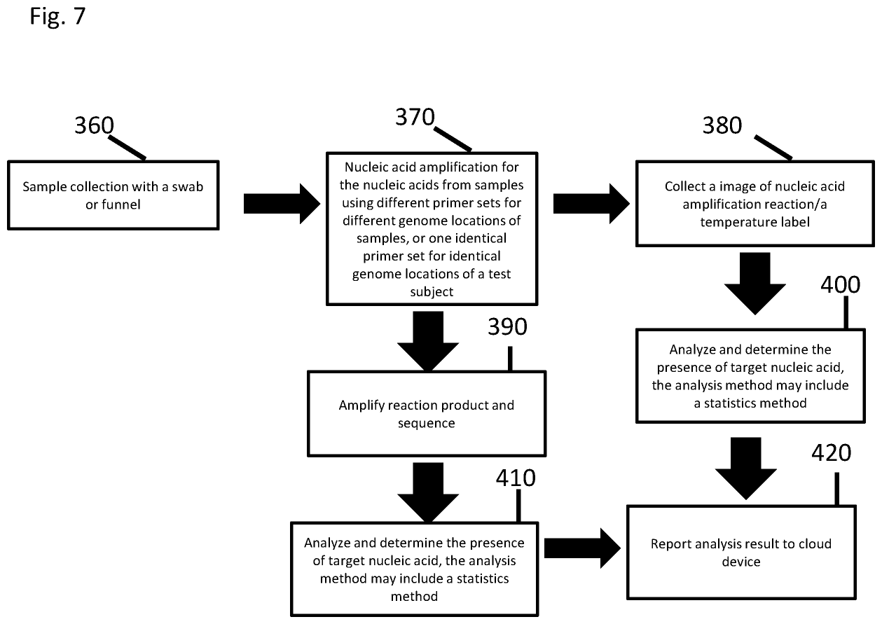

In FIG. 7, the embodiment schematically illustrates the steps for nucleic acid amplification in point of collection at a higher level. The steps begins with collecting a sample from a test subject 360, performing nucleic acid amplification reaction 370, taking an image 380 and analyzing the image and finally determining if target nucleic acid is present in the test subject or not 400. The result will report to a cloud device 420. The analysis method used in 400 may include a statistics method. An exemplary statistics method is t-test or Anova.

Or it can also start with collecting samples 360 and performing nucleic acid amplification 390. The amplified nucleic acid is then sequenced 390. The sequencing data are analyzed and determine presence of target nucleic acid 410. The result will report to a cloud device 420.

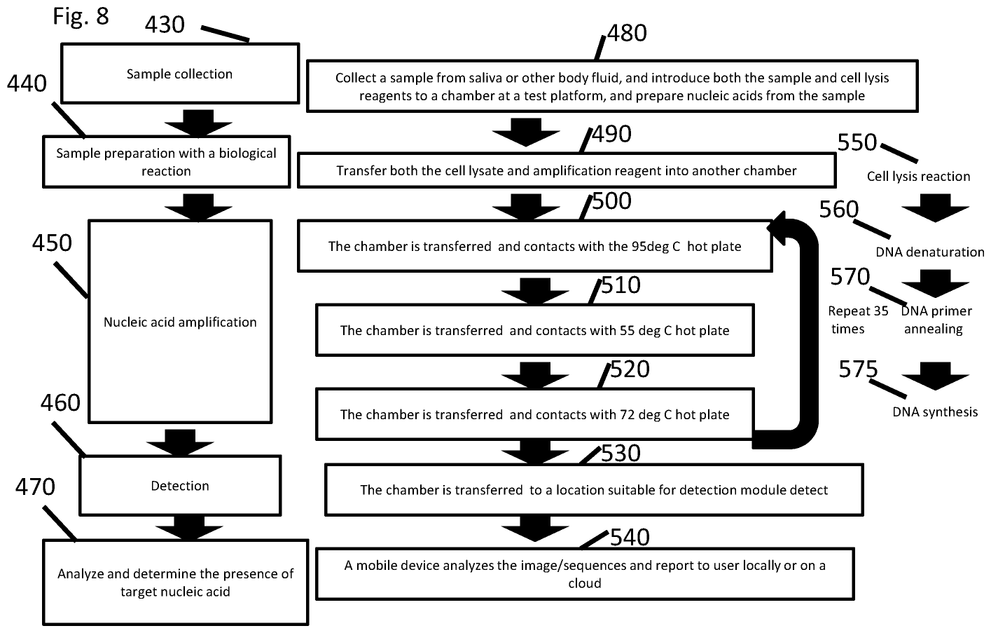

In FIG. 8, the embodiment schematically illustrates the operation steps, procedures and reaction stages of an embodiment method. In terms of the operation stages, the method starts with sample collection 430, sample preparation 440, nucleic acid amplification for the sample 450, detect the nucleic acid amplification product 460, analyze the result to determine if the target nucleic acid is in the sample 470. In terms of procedures, it starts with collecting a sample 480, introducing the sample into a reaction chamber with cell lysis reagents, and then transfer the cell lysate into another reaction chamber 490, keep transferring the reaction chamber and cause it to contact the hot plate in the order of 95 deg C. 500, 55 deg C. 510, 72 deg C. 520, respectively, for a specific time, and repeat the cycle 35 times. Finally, the reaction chamber is moved to a suitable position, the image of nucleic acid amplification product is taken and analyzed 500. The result is 530 then reported to the user or cloud. In terms of reaction, it starts with cell lysis reaction 550 following by DNA denaturation 560, DNA annealing 570 and DNA synthesis 575.

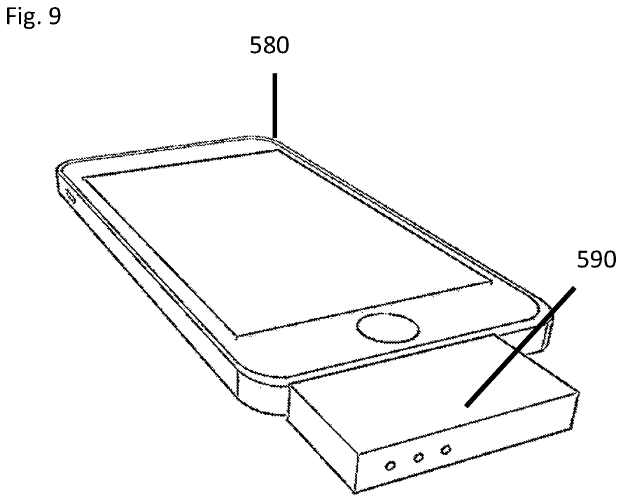

In FIG. 9, the embodiment schematically illustrates a mobile device 580 is adapted with a nanopore sequencer 590, which may sequence the amplified nucleic acid prepared in a reaction chamber of the test platform.

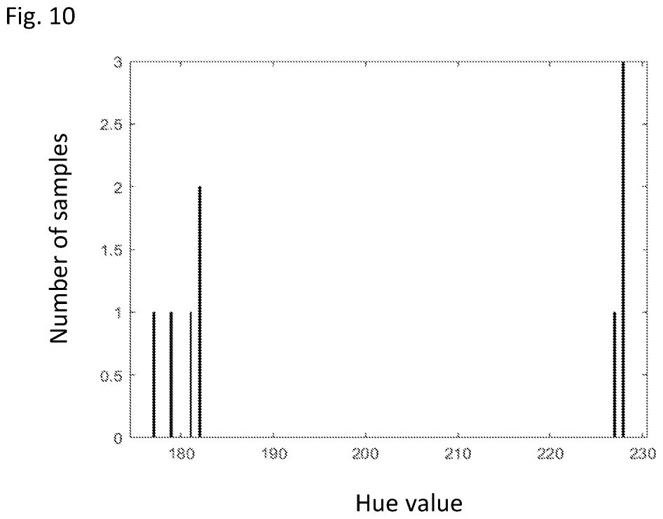

In FIG. 10, the embodiment shows the histogram of hue values obtained from control and treatment samples. The hue values are obtained from the images of PCR products at the control and treatment group. The PCR is performed with the setup described in FIG. 1 and following by the steps-incubate the reaction chamber (a 0.2 ml PCR tube) at 90 deg. for 1 minute, and then start a PCR cycle-95 deg. 20 seconds C, 72 deg. C. 20 seconds, 43 deg. 20 seconds for 45 cycles. The DNA product is generated by amplifying the target nucleic acid 25 ng from M13 phage with 1 uM for both universal M13 forward and reverse primers, and Accuris™ Tag Plus PCR master mix. The 1×SYBR green dye is added for imaging. When imaging, an LED is under the reaction chamber (a 0.2 ml PCR tube) and emits 470 nm light. The control sample has no M13 phage DNA. The hue values are converted from RGB values of images from the treatment and control samples, respectively. The hue values determined herein are: 182, 179, 182, 181, 177 for samples from treatment group (with 25 ng M13 phage DNA) while 224, 227, 228, 228, 228 are for control samples or samples from control group. The p value for one tail t-test is 2.85E-06.

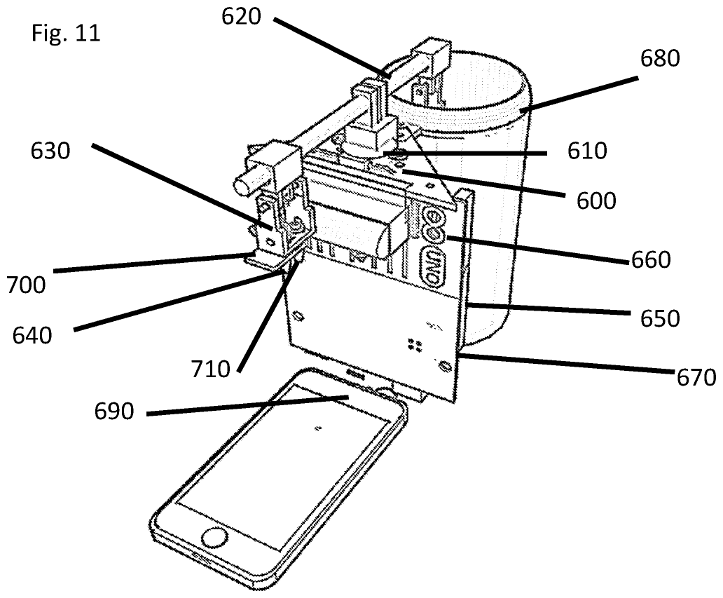

In FIG. 11, the exemplary embodiment shows one electric thermos 680 filled with water and has a predesigned temperature as a heat source. A test platform 600 is supported by the thermos. On the platform, there is a step motor 610 connected to an arm 620. The arm hangs up a receptacle 630. And the receptacle accommodates a tube 640 for a sample. The motor is powered by a battery 650. The position and duration of the arm is controlled by a programmed Arduino UNO R3 board 660 and ULN2003 control board 670. The other tubes 710 contains a control sample and can serve as a color calibration. The temperature label 700 may be used for monitor the temperature of reaction with colorimetric method. By shuttling the arm, the tube may immerse into the thermos—the proximity of a heat source—for a particular period of time. Since the thermo may have a temperature corresponding to isothermal amplification reaction, immersing the tubes into the thermos may change the temperature of nucleic acid amplification reaction, and cause the reaction to complete. Once the reaction is complete, the step motor may drive the arm and move the receptacle out of thermo to a position—a measurement position—that is not over the thermo and suitable a user to take the amplified nucleic acid for nanopore sequencing. Or the measurement position is suitable for a user to measure the color change of product due to the amplified nucleic acid via the camera of a mobile device 690.

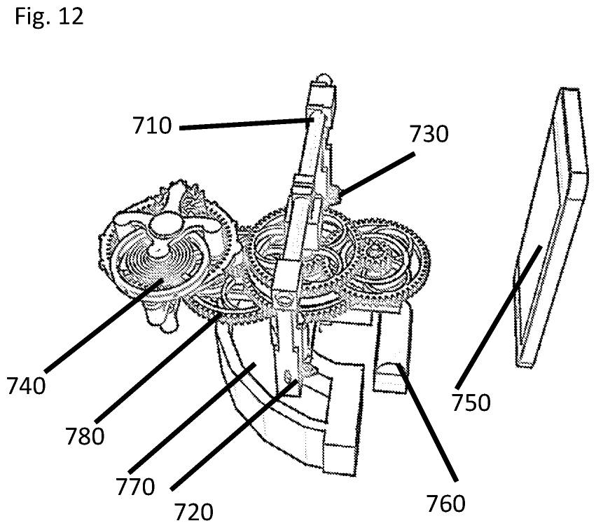

In FIG. 12, a tourbillon is used to shuttle the reaction chambers in a test platform between different heat sources. A clock hand of a tourbillion can serve as an arm 710 and circle around three chemically activated heat sources 770 at three constant temperatures, respectively (as the chemically activated heat source illustrated in FIG. 4). The arm may hang up a receptacle 720 for hosting a reaction chamber 730. The reaction chamber may have a polymerase chain reaction. The reaction chamber may have a thermal contact with the heat sources when the reaction chamber immersed into the water of a heat source. The arm shuttles the reaction chamber to each heat source for each stage of PCR-95 deg. C. for DNA denaturation, 55 deg. C. for primer annealing, 72 deg. C. for DNA synthesis. Once the arm finishes a round, the PCR reaction in the reaction chamber also finish a cycle. The arm can be power by the spring of tourbillion 740 and drives the reaction chamber to the front of a camera of a mobile device 750 by a gear set 780. A LED 760 might be optional for using the colorimetric method to determine amplification result. Thereby, the usage of electricity for nucleic acid amplification in the current disclosure is optional.

### EXEMPLIFICATIONS

Example 1: In this example, as configured in FIG. 1, the system comprises three electric thermos filled with water with the temperatures 95 deg. C., 72 deg. C., 55 deg. C. respectively. One may prepare a DNA sample from a saliva sample (treatment group) by mixing a lysis buffer (i.e. 50 unit/ml Proteinase K with TE buffer) and the saliva in a PCR tube on a floating rack. The tube with the floating rack may be put into the 55 deg. C. thermo for 15 minutes (or follow the method described in Genome Res. 4: 368-370 (1995)) and then 95 deg. C. thermo for 5 minutes as taught in a reference (Rapid and extraction-free detection of SARS-CoV-2 from saliva with colorimetric LAMP, medRxiv. Preprint. 2020 May 11). After the nucleic acid extraction step, the cell lysate as well as PCR master mix and a primer set is then introduced into a PCR tube on the receptacle of a test platform. The test platform sits over three thermos and has an arm driven by a step motor. The arm hangs up the receptacle of tubes (or reaction chambers). By rotating the arm, the tubes (or reaction chambers) may immerse into water in different thermos at each time when the arm moves right above the thermo. Thereby, via rotating the arm, the temperature of reaction in PCR tubes may be controlled. Also, by changing the duration of holding the arm over a particular thermos, the reaction time can be controlled as well. Thereby, by rotating the arm over the thermos with the order: above 95 deg. C. thermo for 15 seconds, 55 deg. C. thermos for 15 second and 55 deg. C. thermos for 15 second, one PCR cycle may be complete. By repeating the same sequence and move the receptacle over the thermos, the nucleic acid amplification reaction may produce enough DNA for colorimetric measurement or DNA sequencing. Once a desired number of PCR cycles is reached, the arm may rotate to a position that is above a cell phone camera. One may add SYBR Green dye into the reaction, and turn on a 395 nm LED light beneath the tube. An image for both the tube and color calibration is taken by the camera on a cell phone. The control sample (control group, which may serve as a color calibration as well) is another tube with the PCR reagents, primers set and SYBR Green dye but DNA from saliva sample (treatment group). The image is processed by retrieving the RGB values from both tubes from the treatment and control group (or a color calibration). The RGB values are then converted to hue values. If the difference of hue values between the saliva sample (treatment group) and control sample (control group) is above a threshold, the target DNA may present in the saliva sample.

The DNA produced in this way can be collect and preserve for a portable nucleic acid sequencer such as a nanopore sequencer. Following the procedure instructed in (Multiplex PCR method for MinION and Illumina sequencing of Zika and other virus genomes directly from clinical samples, Nature Protocols, 12, 1261 (2017)), one may sequence the amplified DNA and processed the sequencing data from a mobile device. Thereby, a genome sequencing information may be obtained in a point-of-collection manner. Since the thermo with a temperature control can be easily obtained at a low cost, and the thermo may be used for drink or other beverage after all. The current disclosure is particularly suitable for location with a very limited resource. Furthermore, in term of period of duration for completing one PCR cycle, the current disclosure requires less a than half of time than a convention thermal cycler for a 100 bp DNA synthesis, which requires heating up or cooling down a heating block before reaching a predesigned temperature.

Example 2: In one embodiment, a carrier may have four wheels and is able to move linearly. A receptacle may sit in the carrier. The receptacle is able to accommodate 1600 reaction chambers and has an area of 60 cm×60 cm. In one embodiment, the samples may be collected by capillaries shown in FIG. 5. and introduced into the receptacle. In one embodiment, the receptacle may have pre-dry primer set and/or wax beads with PCR master mix before the samples are introduced. In one embodiment, the primer sets may cover different genome locations of one or more organisms. In one embodiment, the receptacle may also be able to contact with the top surface of a silicon rubber heater. There may be three silicon rubber heaters, and each may have a top surface area 70 cm×70 cm with preset temperature 95 deg. C., 72 deg. C., 55 deg. C. respectively. These silicon rubber heaters may be aligned in a line so that the carrier may move over them in a direction. The carried is driven by motors and may translocate the receptacle over each silicon rubber heater for each predetermined time, and complete the nucleic acid amplification reaction after a certain number of PCR cycles. Thereby, the 1600 nucleic acid amplification reactions can all be complete at once. Since a silicon rubber heater is easy to pack and carry, and a mobile device is easy to access, the current disclosure is particularly useful in certain locations. Such as a farm or a remote area, sometime, a large number of nucleic acid reactions needs to be carried out but a high throughput facility is not available.

Example 3: In one embodiment, plurality of reaction chambers may contain identical primer sets. Thereby, plurality of identical reactions may be carried out under the same conditions. If there are three or more samples collected from each a control group and a treatment group, respectively, a proper statistics method such as t-test or Analysis of variance (ANOVA) can be used to determine the confidence level of results. Since each hue value can be obtained from the colorimetric measurements of each reaction, the hue values may be used to determine if a null hypothesis—the samples from control group are identical to the samples from treatment group—is valid under certain confidence level such as p value below 0.05.

Example 4: In one embodiment, a temperature label can associate with a heat source or a reactive test region, and the temperature label changes color when the temperature of heat source or reactive test region changes. Thereby, the temperature of a heat source or reactive test region is monitored via images taken by a mobile device. The mobile device can be installed with software. The software can process the images for the color coordinates and determine the temperature of the heat source or reactive test region.

Example 5: In one embodiment as shown in FIG. 12, a tourbillon is used to shuttle the reaction chambers in a test platform between different heat sources. A clock hand of a tourbillion can serve as an arm and circle around three chemically activated heat sources at three different temperatures (as the chemically activated heat source illustrated in FIG. 4). The arm may hang up a receptacle for hosting a reaction chamber. The reaction chamber may have a polymerase chain reaction. The reaction chamber may have a thermal contact with each heat source at once when the arm shuttles the reaction chamber for each different amplification stage. Each time, when the arm of tourbillion completes a round, one PCR cycle can be complete as well. Thereby, the usage of electricity in current disclosure is optional.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

1. A system for processing a sample, the system comprising:
at least one heat source;
at least one reaction chamber on a test platform;
nucleic acid amplification reaction reagents reacting with said sample through nucleic acid amplification reactions to produce amplified nucleic acid;
a mobile device having a detection module and installed software; and
means to shuttle said reaction chamber to a position;
wherein said position corresponds to either the proximity of said at least one heat source or a measurement position for said nucleic acid amplification reactions;
wherein said measurement position is suitable for said detection module to perform measurement or collect amplified nucleic acid for measurement;
wherein said measurement is either to take an image of said nucleic acid amplification reactions or to sequence said amplified nucleic acid;
wherein said mobile device is configured in a manner to quantify and/or sequence said amplified nucleic acid;
wherein said at least one heat source has thermal communication with said at least one reaction chamber;
wherein said means shuttles said at least one reaction chamber to control the temperature and duration of said nucleic acid amplification reaction, or to a suitable position for quantifying and/or sequencing said amplified nucleic acid via said mobile device;
wherein said installed software processes the image taken by said detection module or sequencing data generated by said detection module.

2. The system of claim 1, wherein said at least one heat source comprises either a chemically activated heating material or a container with an electric thermal stat.

3. The system of claim 1, wherein said means may be a combination of one or more of arms, linkages, belts or similar facilities that cause said at least one reaction chamber to have thermal communication with at least one heat source.

4. The system of claim 1, wherein said detection module is a nanopore sequencer.

5. The system of claim 1, wherein said installed software implemented a t-test method or analysis of variance method to determine whether samples in a control group are different from samples in a treatment group within a confidential level.

6. The system of claim 1, wherein said detection module is a camera of said mobile device.

7. The system of claim 1, wherein said at least one reaction chamber is a capillary with at least one closed end.

8. The system of claim 1, wherein said detection module comprises an LED light source.

9. The system of claim 1, wherein said test platform comprises a color calibration or a temperature label.

10. The system of claim 1, wherein said nucleic acid amplification reaction reagents are either lyophilized or stored in a wax.

11. A method for processing a sample, the method comprising the steps of: providing (i) a test platform that includes at least one reaction chamber to receive said sample and nucleic acid amplification reaction reagents, wherein said sample and said nucleic acid amplification reaction agents cause nucleic acid amplification reaction to produce amplified nucleic acid;
(ii) a plurality of heat sources;
(iii) at least one mobile device with a detection module for said amplified nucleic acid;
(iv) means to shuttle said at least one reaction chamber to different positions; wherein said positions are adjacent to either said plurality of heat sources or other positions suitable for taking an image of said nucleic acid amplification reaction or collection of said amplified nucleic acid;
introducing said sample into said at least one reaction chamber;
sealing said at least one reaction chamber;
controlling the temperature of said reaction chamber for said nucleic acid amplification reaction via shuttling said reaction chamber to the proximity of said plurality of heat sources or between proximity of said plurality of heat sources and said detection module, wherein said plurality of heat sources each has a particular temperature, wherein said plurality of heat sources has thermal communication with said at least one reaction chamber when said at least one reaction chamber is adjacent to said plurality of heat sources, thereby the temperature of said at least one reaction chamber is controlled by moving said at least one reaction chamber adjacent to one of said plurality of heat sources having a particular temperature, wherein a duration of said temperature of said at least one reaction chamber is controlled by holding said at least one reaction chamber adjacent to said one of the plurality of heat sources for a specific period of time; and
detecting said amplified nucleic acid via shuttling said reaction chamber to a suitable position for said mobile device to take at least one image of said nucleic acid amplification reaction or sequencing said amplified nucleic acid via shuttling said reaction chamber to a suitable position for collection of said amplified nucleic acid for sequencing by said detection module of said mobile device.

12. The method of claim 11, wherein said image is used to quantify said nucleic acid amplification reaction with colorimetric method.

13. The method of claim 11, wherein said sample is prepared with one of said plurality of heat sources.

14. The method of claim 11, wherein said mobile device may transmit said image or said sequencing result to a cloud device.

15. The method of claim 11, wherein the step of sealing said at least one reaction chamber is performed with a grease, wax, or sealant.

16. The method of claim 11, wherein the step of introducing said sample into said at least one reaction chamber is performed through at least one capillary via either capillary action or suction of a suction device.

17. The method of claim 11, wherein said nucleic acid amplification reaction is a polymerase chain reaction or nucleic isothermal amplification reaction.

18. A method for processing a plurality of nucleic acid amplification reactions in a point-of-collection manner, the method comprising the steps of:
providing (i) at least one heat source; (ii) at least one receptacle to accommodate the plurality of nucleic acid amplification reactions; (iii) a mobile device having a detection module; (vi) one or more test subjects; and (v) means to shuttle said at least one receptacle;
introducing nucleic acid from said one or more test subjects;
shuttling said at least one receptacle to the proximity of said at least one heat source to cause said plurality of nucleic acid amplification reactions to complete;
shuttling said at least one receptacle to the proximity of said detection module for a suitable position for quantifying amplified nucleic acids produced from said plurality of nucleic acid amplification reactions;
quantifying said amplified nucleic acids to obtain a plurality of nucleic acid amplification reaction results;
performing analysis of said plurality of nucleic acid amplification reaction results; and
reporting said results on said mobile device or transmit said results to a cloud device.

19. The method of claim 18, wherein said plurality of nucleic acid amplification reactions use different primer sets for different genome locations of the same test subject.

20. The method of claim 18, wherein said plurality of nucleic acid amplification reactions use one identical primer set for identical genome locations of a test subject.
